(19) United States
(12) Patent Application Publication
(10) Pub. No.: US 2011/0008864 A1
(43) Pub. Date: Jan. 13, 2011

(54) Processes for Producing Fermentation Products

(75) Inventors: Randy Deinhammer, Wake Forest, NC (US); Guillermo Coward-Kelly, Wake Forest, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc., Franklinton, NC (US)

(21) Appl. No.: 12/922,060

(22) PCT Filed: Mar. 30, 2009
(86) PCT No.: PCT/US09/38785

§ 371 Date: Sep. 10, 2010

Related U.S. Application Data
(60) Provisional application No. 61/040,166, filed on Mar. 28, 2008.

(51) Int. Cl. C12P 7/10; C12P 1/00; C12P 7/06

(52) U.S. Cl. 435/165; 435/41; 435/161

(57) ABSTRACT

The invention relates to a process of fermenting plant material in a fermentation medium into a fermentation product using a fermenting organism, wherein one or more trehalases are present in the fermentation medium.

### TECHNICAL FIELD

The present invention relates to methods of fermenting plant derived material into desired fermentation products. The invention also relates to processes of producing a fermentation product from plant material using one or more fermenting organisms; compositions; transgenic plants; and modified fermenting organisms, that can be used in methods and/or processes of the invention.

### BACKGROUND ART

A vast number of commercial products that are difficult to produce synthetically are today produced by fermenting organisms. Such products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); and hormones. Fermentation is also commonly used in the consumable alcohol (e.g., beer and wine), dairy (e.g., in the production of yogurt and cheese), leather, and tobacco industries.

A vast number of processes of producing fermentation products, such as ethanol, by fermentation of sugars provided by degradation of starch-containing and/or lignocellulose-containing material are known in the art.

However, production of fermentation products, such as ethanol, from such plant materials is still too costly. Therefore, there is a need for providing processes that can increase the yield of the fermentation product and thereby reducing the production costs.

### SUMMARY OF THE INVENTION

The present invention relates to methods of fermenting plant derived material (i.e., fermentable sugars) into a fermentation product. The invention also provides processes of producing fermentation products from plant material using one or more fermenting organisms. Furthermore, the invention relates to compositions comprising one or more trehalases, which compositions are suitable for use in methods and processes of the invention. Finally the invention relates to transgenic plants and modified fermenting organisms.

According to the invention the starting material (i.e., substrate for the fermenting organism in question) may be any plant material, especially plant derived material. The material may be treated and/or untreated. The stating material may in one embodiment be starch-containing material. The starting material may in another embodiment be lignocellulose-containing material.

In the first aspect the invention relates to methods of fermenting sugars derived from plant material in a fermentation medium into a fermentation product using one or more fermenting organisms, wherein one or more trehalases are present in the fermentation medium. According to the invention the concentration/dose level of fermentation product, such as ethanol, is increased compared to a corresponding method when no such trehalase is present or added before and/or during fermentation.

In the second aspect the invention relates to processes of producing a fermentation product from starch-containing material comprising the steps of:

i) liquefying starch-containing material;

ii) saccharifying the liquefied material; and

iii) fermenting with one or more fermenting organisms,

wherein fermentation is carried out in the presence of one or more trehalases.

In a third aspect the invention relates to processes of producing a fermentation product from starch-containing material comprising the steps of:

i) liquefying starch-containing material;
ii) saccharifying the liquefied material;
iii) fermenting with one or more fermenting organisms,
wherein backset subjected to trehalase treatment is adding during and/or before step i).

In a fourth aspect the invention relates to processes of producing a fermentation product from starch-containing material, comprising the steps of:

(a) saccharifying starch-containing material at a temperature below the initial gelatinization temperature of said starch-containing material; and

(b) fermenting using one or more fermenting organisms, wherein fermentation is carried out in the presence of one or more trehalases.

It is to be understood that in the fourth aspect of the invention the starch-containing material is not subjected to liquefaction, such as a conventional liquefaction step, as no substantial gelatinization of the starch material takes place.

In a fifth aspect the invention relates to processes of producing a fermentation product from lignocellulose-containing material, comprising the steps of:

(a) pre-treating the lignocellulose-containing material;

(b) hydrolysing the material;

(c) fermenting using one or more fermenting organisms,

wherein fermentation is carried out in the presence of one or more trehalases.

In a fifth aspect the invention relates to processes of producing a fermentation product comprising:

i) fermenting plant material derived sugars using one or more fermenting organisms;
ii) treating the fermenting plant material with one or more trehalases;
ii) recycling of trehalase treated material to step i).

In the sixth aspect the invention relates to a composition comprising one or more trehalases.

In the seventh aspect the invention relates to the use of trehalase or compositions of the invention in a fermentation method or process of the invention.

In the eighth aspect the invention relates to a transgenic plant material, wherein plant material has been transformed with a polynucleotide sequence encoding a trehalase.

In the ninth aspect the invention relates to modified fermenting organisms, wherein fermenting organisms have been transformed with a polynucleotide encoding a trehalase, wherein the fermenting organism is capable of expressing trehalase at fermentation conditions.

### BRIEF DESCRIPTION OF THE FIGURES

FIG. 1 shows the effect of adding trehalase to liquefied corn mash from Adkins Energy after 0, 24 and 48 hours fermentation;

FIG. 2 shows the effect of adding trehalase to liquefied corn mash from Verasun Energy after 0 and 63 hours fermentation;

FIG. 3 shows a chromatogram from an ion chromatographic system which illustrates the reduction in the peak for trehalose observed upon addition of trehalase to corn mash (Verasun Energy) fermentation;

FIG. 4 shows the effect of using trehalase on backset fermentation.

### DETAILED DESCRIPTION OF THE INVENTION

Trehalose is a stable disaccharide sugar consisting of two sugar monomers (glucose). Trehalose is accumulated in yeast as a response to stress in up to 10-15% of cell dry weight (GrBa et al. (1975) Eur. J. Appl. Microbiol. 2:29-37).

Without being bound by any particular theory, the present invention provides the addition of one or more trehalase enzymes capable of hydrolyzing extracellular trehalose into two molecules of glucose to fermentation processes before and/or during fermentations using one or more fermenting organisms. The resulting glucose can then be converted into a desired fermentation product, such as ethanol, by fermenting organisms, such as yeast, resulting in an increased fermentation product yield.

Consequently, in the first aspect the invention relates to methods of fermenting sugars derived from plant material in a fermentation medium into a fermentation product using one or more fermenting organisms, wherein one or more trehalases are present in the fermentation medium.

The trehalase(s) may be added/introduced before and/or during fermentation and/or may be produced, e.g., in situ by over-expression of trehalase by the fermenting organism(s), preferably yeast. This can be accomplished by preparing modified fermenting organisms, e.g., yeast, that are capable of expressing trehalase, e.g., by transformation of one or more trehalase encoding genes or by introducing a stronger promoter that increases expression of the trehalase gene(s) already present in the fermenting organism(s). In a preferred embodiment the trehalase is secreted into the fermenting medium. Techniques for introducing a trehalase gene(s) into fermenting organisms, such as yeast, and/or over-expressing trehalase genes in fermenting organisms are known in the art. Trehalase(s) may also be present/introduced into the fermentation medium in the form of transgenic plant material containing and/or expressing trehalase(s).

### Trehalases

Trehalases are enzymes which degrade trehalose into its unit monosaccharides (i.e., glucose). According to the invention trehalase may be one single trehalase, or a combination of two of more trehalases of any origin, such as plant, mammalian, or microbial origin, such a bacterial or fungal origin. In a preferred embodiment the trehalase is of mammalian origin, such as porcine trehalase. In another preferred embodiment the trehalase is of fungal origin, preferably of yeast origin. In a preferred embodiment the trehalase is derived from a strain of Saccharomyces, such as a strain of Saccharomyces cervisae.

Trehalases are classified in EC 3.2.1.28 (alpha,alpha-trehalase) and EC. 3.2.1.93 (alpha,alpha-phosphotrehalase). The EC classes are based on recommendations of the Nomenclature Committee of the International Union of Biochemistry and Molecular Biology (IUBMB). Description of EC classes can be found on the internet, e.g., on “http://www.expasy.org/enzyme/”. Trehalases are enzymes that catalyze the following reactions:

### EC 3.2.1.28:

Alpha,alpha-trehalose+H₂O=2 D-glucose;

### EC 3.2.1.93:

Alpha,alpha-trehalose 6-phosphate+H₂O<=>D-glucose+D-glucose 6-phosphate;

The two enzyme classes are both referred to as “trehalases” in context of the present invention. In a preferred embodiment the trehalase is classified as EC 3.2.1.28. In another embodiment the trehalase is classified as EC 3.2.1.93. In embodiment the trehalase is a neutral trehalase. In another embodiment the trehalase is an acid trehalase.

Examples of neutral trehalases include, but are not limited to, trehalases from Saccharomyces cerevisiae (Londesborouh et al. (1984) Characterization of two trehalases from baker's yeast” Biochem J 219, 511-518; Mucor roxii (Dewerchin et al (1984), “Trehalase activity and cyclic AMP content during early development of Mucor rouxii spores”, J. Bacteriol. 158, 575-579); Phycomyces blakesleeanus (Thevelein et al (1983), “Glucose-induced trehalase activation and trehalose mobilization during early germination of Phycomyces blakesleeanus spores” J. Gen Microbiol. 129, 719-726); Fusarium oxysporium (Amaral et al (1996), “Comparative study of two trehalase activities from Fusarium oxysporium var Linii” Can. J Microbiol. 41, 1057-1062);

Examples of neutral trehalases include, but are not limited to, trehalases from Saccharomyces cerevisiae (Parvaeh et al. (1996) Purification and biochemical characterization of the ATH1 gene product, vacuolar acid trehalase from Saccharomyces cerevisae” FEBS Lett. 391, 273-278); Neorospora crassa (Hecker et al (1973), “Location of trehalase in the 30 ascospores of Neurospora: Relation to ascospore dormancy and germination”. J. Bacteriol. 115, 592-599); Chaetomium aureum (Sumida et al. (1989), “Purification and some properties of trehalase from Chaetomium aureum MS-27. J. Ferment. Bioeng. 67, 83-86); Aspergillus nidulans (d'Enfert et al. (1997), “Molecular characterization of the Aspergillus nidulans treA gene encoding an acid trehalase required for growth on trehalose. Mol. Microbiol. 24, 203-216); Humicola grisea (Zimmermann et al. (1990).” Purification and properties of an extracellular conidial trehalase from Humicola grisea var. thermoidea”, Biochim. Acta 1036, 41-46); Humicola grisea (Cardello et al. (1994), “A cytosolic trehalase from the thermophilhilic fungus Humicola grisea var. thermoidea, Microbiology UK 140, 1671-1677; Scytalidium thermophilum (Kadowaki et al. (1996), “Characterization of the trehalose system from the thermophilic fungus Scytalidium thermophilum” Biochim. Biophys. Acta 1291, 199-205); and Fusarium oxysporium (Amaral et al (1996), “Comparative study of two trehalase activities from Fusarium oxysporium var Linii”Can. J Microbiol. 41, 1057-1062).

A trehalase is also know from soybean (Aeschbachet et al (1999) “Purification of the trehalase GmTRE1 from soybean nodules and cloning of its cDNA”, Plant Physiol 119, 489-496).

Trehalases are also present in small intestine and kidney of mammals. Commercially available trehalase includes the porcine trehalase available from SIGMA, USA (product # A8778).

The trehalase may be added or present in any effective dosage during fermentation, which includes, but is not limited to, from 1 to 500 Sigma units per liter fermentation medium, preferably 10-100 Sigma units per liter fermentation medium.

### Fermenting Organisms

The term “fermenting organism” refers to any organism, including bacterial and fungal organisms, including yeast and filamentous fungi, suitable for producing a desired fermentation product. The fermenting organism may be C6 or C5 fermenting organisms, or a combination thereof. Both C6 and C5 fermenting organisms are well known in the art.

Suitable fermenting organisms according to the invention are able to ferment, i.e., convert fermentable sugars, such as glucose, fructose maltose, xylose, mannose or arabinose, directly or indirectly into the desired fermentation product.

Examples of fermenting organisms include fungal organisms such as yeast. Preferred yeast includes strains of the genus Saccharomyces, in particular strains of Saccharomyces cerevisiae or Saccharomyces uvarum; a strain of Pichia, preferably Pichia stipitis such as Pichia stipitis CBS 5773 or Pichia pastoris; a strain of the genus Candida, in particular a strain of Candida utilis, Candida arabinofermentans, Candida diddensii, Candida sonorensis, Candida shehatae, Candida tropicalis, or Candida boidinii. Other fermenting organisms include strains of Hansenula, in particular Hansenula polymorpha or Hansenula anomala; Kluyveromyces, in particular Kluyveromyces fragilis or Kluyveromyces marxianus; and Schizosaccharomyces, in particular Schizosaccharomyces pombe.

Preferred bacterial fermenting organisms include strains of Escherichia, in particular Escherichia coli, strains of Zymomonas, in particular Zymomonas mobilis, strains of Zymobacter, in particular Zymobactor palmae, strains of Klebsiella in particular Klebsiella oxytoca, strains of Leuconostoc, in particular Leuconostoc mesenteroides, strains of Clostridium, in particular Clostridium butyricum, strains of Enterobacter, in particular Enterobacter aerogenes and strains of Thermoanaerobacter, in particular Thermoanaerobacter BG1L1 (Appl. Microbiol. Biotech. 77: 61-86) and Thermoanarobacter ethanolicus, Thermoanaerobacter thermosaccharolyticum, or Thermoanaerobacter mathranii. Strains of Lactobacillus are also envisioned as are strains of Corynebacterium glutamicum R, Bacillus thermoglucosidaisus, and Geobacillus thermoglucosidasius.

In an embodiment the fermenting organism is a C6 sugar fermenting organism, such as a strain of, e.g., Saccharomyces cerevisiae.

In connection with fermentation of lignocellulose derived materials, C5 sugar fermenting organisms are contemplated. Most C5 sugar fermenting organisms also ferment C6 sugars. Examples of C5 sugar fermenting organisms include strains of Pichia, such as of the species Pichia stipitis. C5 sugar fermenting bacteria are also known. Also some Saccharomyces cerevisae strains ferment C5 (and C6) sugars. Examples are genetically modified strains of Saccharomyces spp. that are capable of fermenting C5 sugars include the ones concerned in, e.g., Ho et al., 1998, Applied and Environmental Microbiology, p. 1852-1859 and Karhumaa et al., 2006, Microbial Cell Factories 5:18, and Kuyper et al., 2005, FEMS Yeast Research 5: 925-934.

Yeast is a preferred fermenting organism for ethanol fermentation. Preferred are strains of Saccharomyces, especially strains of the species Saccharomyces cerevisiae, preferably strains which are resistant towards high levels of ethanol, i.e., up to, e.g., about 10, 12, 15 or 20 vol. % or more ethanol.

In one embodiment the fermenting organism is added to the fermentation medium so that the viable fermenting organism, such as yeast, count per mL of fermentation medium is in the range from 10⁵ to 10¹², or from 10⁷ to 10¹⁰, or about 5×10⁷.

Commercially available yeast includes, e.g., RED START™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties).

According to the invention the fermenting organism capable of producing a desired fermentation product from fermentable sugars, such as, e.g., glucose, fructose maltose, xylose and/or arabinose, is preferably grown under precise conditions at a particular growth rate. When the fermenting organism(s) is(are) introduced into/added to the fermentation medium the inoculated fermenting organism(s) pass through a number of stages. Initially growth does not occur. This period is referred to as the “lag phase” and may be considered a period of adaptation. During the next phase referred to as the “exponential phase” the growth rate gradually increases. After a period of maximum growth the rate ceases and the fermenting organism(s) enter(s) “stationary phase”. After a further period of time the fermenting organism(s) enter(s) the “death phase” where the number of viable cells declines.

In one embodiment the trehalase(s) are added to the fermentation medium when the fermenting organism(s) is(are) in lag phase.

In one embodiment trehalase(s) are added to the fermentation medium when the fermenting organism(s) is(are) in exponential phase.

In one embodiment trehalase(s) are added to the fermentation medium when the fermenting organism(s) is(are) in stationary phase.

### Fermentation Products

The term “fermentation product” means a product produced by a method or process, including a fermentation step, using one or more fermenting organisms. Fermentation products contemplated according to the invention include alcohols (e.g., ethanol, methanol, butanol); organic acids (e.g., citric acid, acetic acid, itaconic acid, lactic acid, succinic acid, gluconic acid);

ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferred fermentation processes used include alcohol fermentation processes. The fermentation product, such as ethanol, obtained according to the invention, may preferably be used as fuel. However, in the case of ethanol it may also be used as potable ethanol.

### Fermentation Medium

The term “fermentation medium” refers to the environment in which fermentation is carried out and which includes the fermentable substrate, that is, a carbohydrate source that can be metabolized by the fermenting organism(s).

The fermentation medium may comprise nutrients and/or growth stimulator(s) for the fermenting organism(s). Nutrient and growth stimulators are widely used in the art of fermentation and include nitrogen sources, such as ammonia; vitamins; and minerals, or combinations thereof.

### Fermentation

The plant starting material used in fermenting methods or processes of the invention may be starch-containing material and/or lignocellulose-containing material. The fermentation conditions are determined based on, e.g., the kind of plant material, the available fermentable sugars, the fermenting organism(s) and/or the desired fermentation product. One skilled in the art can easily determine suitable fermentation conditions. The fermentation may according to the invention be carried out at conventionally used conditions. Preferred fermentation processes are anaerobic processes.

The methods or processes of the invention may be performed as batch or as continuous processes. Fermentations of the invention may be conducted in an ultrafiltration system where the retentate is held under recirculation in the presence of solids, water, and the fermenting organism(s), and where the permeate is the desired fermentation product containing liquid. Equally contemplated is methods/processes conducted in continuous membrane reactors with ultrafiltration membranes and where the retentate is held under recirculation in presence of solids, water, the fermenting organism(s) and where the permeate is the fermentation product containing liquid.

After fermentation the fermenting organism(s) may be separated from the fermented slurry and recycled.

### Fermentation of Starch-Derived Sugars

Different kinds of fermenting organisms may be used for fermenting sugars derived from starch-containing material. Fermentations are conventionally carried out using yeast, such as Saccharomyces cerevisae, as the fermenting organism. However, bacteria and filamentous fungi may also be used as fermenting organisms. Some bacteria have higher fermentation temperature optimum than, e.g., Saccharomyces cerevisae. Therefore, fermentations may in such cases be carried out at temperatures as high as 75° C., e.g., between 40-70° C., such as between 50-60° C. However, bacteria with a significantly lower temperature optimum down to around room temperature (around 20° C.) are also known. Examples of suitable fermenting organisms can be found in the “Fermenting Organisms” section above.

For ethanol production using yeast, the fermentation may in one embodiment go on for 24 to 96 hours, in particular for 35 to 60 hours. In an embodiment the fermentation is carried out at a temperature between 20 to 40° C., preferably 26 to 34° C., in particular around 32° C. In an embodiment the pH is from pH 3 to 6, preferably around pH 4 to 5.

Other fermentation products may be fermented at temperatures known to the skilled person in the art to be suitable for the fermenting organism in question.

Fermentations are typically carried out at a pH in the range between 3 and 7, preferably from pH 3.5 to 6, such as around pH 5. Fermentations are typically ongoing for 24-96 hours.

### Fermentation of Lignocellulose-Derived Sugars

Different kinds of fermenting organisms may be used for fermenting sugars derived from lignocellulose-containing materials. Fermentations are typically carried out by yeast, bacteria or filamentous fungi, including the ones mentioned in the “Fermenting Organisms”-section above. If the aim is C6 fermentable sugars the conditions are usually similar to starch fermentations as described above. However, if the aim is to ferment C5 sugars (e.g., xylose) or a combination of C6 and C5 fermentable sugars the fermenting organism(s) and/or fermentation conditions may differ.

Bacteria fermentations may be carried out at higher temperatures, such as up to 75° C., e.g., between 40-70° C., such as between 50-60° C., than conventional yeast fermentations, which are typically carried out at temperatures from 20-40° C. However, bacteria fermentations at temperature as low as 20° C. are also known. Fermentations are typically carried out at a pH in the range between 3 and 7, preferably from pH 3.5 to 6, such as around pH 5. Fermentations are typically ongoing for 24-96 hours.

### Recovery

Subsequent to fermentation the fermentation product may be separated from the fermentation medium. The fermentation medium may be distilled to extract the desired fermentation product or the desired fermentation product may be extracted from the fermentation medium by micro or membrane filtration techniques. Alternatively, the fermentation product may be recovered by stripping. Methods for recovery are well known in the art.

### Thin Stillage/Backset

After fermentation most starch has been used/converted. As mentioned above (liquid) fermentation products, such as ethanol, are typically recovered from the fermented mash (often referred to as “beer mash”), e.g., by distillation, which separates the desired fermentation product from other liquids and/or solids. The remaining faction, referred to as “whole stillage,” is dewatered and separated into a solid and a liquid phase, e.g., by centrifugation. The solid phase is referred to as “Wet Distiller's Grains” (or “wet cake”) and the liquid phase (supernatant) is referred to as “thin stillage.” Wet distiller's grains (WDG) is dried to provide “distiller's dried grain” (DDG) used as nutrient in animal feed. Thin stillage is typically evaporated to provide condensate and syrup or may alternatively be recycled directly to a slurry tank as “backset.” Condensate may be forwarded to a methanator before being discharged or recycled to a slurry tank. The syrup consisting mainly of limit dextrins and non-fermentable sugars may be blended into DDG or added to the Wet Distiller's Grains before drying to produce DDG/S (Distillers Dried Grains with Solubles).

According to the invention backset may be recycled to the process of the invention.

Production of Fermentation Products from Starch-Containing Material

### Processes for Producing Fermentation Products From Gelatinized Starch-Containing Material

In this aspect the present invention relates to processes for producing a fermentation product, especially ethanol, from starch-containing material, which process includes a liquefaction step, and sequentially or simultaneously performed saccharification and fermentation steps.

The invention relates to a process for producing a fermentation product from starch-containing material comprising the steps of:

i) liquefying starch-containing material;

ii) saccharifying the liquefied material;

iii) fermenting using one or more fermenting organisms,

wherein fermentation is carried out in the presence of one or more trehalases.

In one embodiment, the trehalase is added before fermentation, preferably during saccharification in step ii).

As mentioned above backset may according to the invention be recycled to a suitable process step in a process of the invention. Typically the backset is recycled to the slurry prepared before liquefaction in step i).

Therefore, the invention also relates to processes of producing a fermentation product from starch-containing material comprising the steps of:

i) liquefying starch-containing material;
ii) saccharifying the liquefied material;
iii) fermenting with one or more fermenting organisms,
wherein backset subjected to trehalase treatment is adding during and/or before step i).

In another embodiment trehalase is added after liquefaction, preferably to saccharification in step ii).

In a particular embodiment, the process of the invention further comprises, prior to the step i), the steps of:

x) reducing the particle size of the starch-containing material, preferably by milling;

y) forming a slurry comprising the starch-containing material and water.

In an embodiment backset subjected to trehalase treatment is added to the slurry in step y).

The aqueous slurry may contain from 10-55 wt. % dry solids (DS), preferably 25-45 wt. % dry solids (DS), more preferably 30-40wt. % dry solids (DS) of starch-containing material. The slurry is heated to above the gelatinization temperature and alpha-amylase, preferably bacterial and/or acid fungal alpha-amylase may be added to initiate liquefaction (thinning). The slurry may in an embodiment be jet-cooked to further gelatinize the slurry before being subjected to an alpha-amylase in step i) of the invention.

Saccharification step ii) and fermentation step iii) may be carried out either sequentially or simultaneously. The trehalase(s) may be added before (e.g., during liquefaction step i) or separate saccharification step ii)) and/or during the fermentation step iii) or simultaneous saccharification and fermentation step.

The desired fermentation product, such as especially ethanol, may optionally be recovered after fermentation, e.g., by distillation. Suitable starch-containing starting materials are listed in the section “Starch-Containing Materials” section below. Contemplated enzymes are listed in the “Enzymes” section below. The liquefaction is preferably carried out in the presence of at least an alpha-amylase, preferably a bacterial alpha-amylase and/or acid fungal alpha-amylase. The fermenting organism is preferably yeast, preferably a strain of Saccharomyces cerevisiae. However, other suitable fermenting organisms are listed in the “Fermenting Organisms” section above.

Liquefaction may be carried out as a three-step hot slurry process. The slurry is heated to between 60-95° C., preferably 80-85° C., and alpha-amylase is added to initiate liquefaction (thinning). Then the slurry may be jet-cooked at a temperature between 95-140° C., preferably 105-125° C., for about 1-15 minutes, preferably for about 3-10 minutes, especially around about 5 minutes. The slurry is cooled to 60-95° C. and more alpha-amylase is added to finalize hydrolysis (secondary liquefaction). The liquefaction process is usually carried out at pH 4.5-6.5, in particular at a pH from 5 to 6.

The saccharification step (ii) may be carried out using conditions well know in the art. For instance, a full saccharification step may last up to from about 24 to about 72 hours, however, it is also common only to do a pre-saccharification of typically 40-90 minutes at a temperature between 30-65° C., typically about 60° C., followed by complete saccharification during fermentation in a simultaneous saccharification and fermentation step (SSF) process.

Saccharification is typically carried out at temperatures from 20-75° C., preferably from 40-70° C., typically around 60° C., and at a pH between about 4 and 5, normally at about pH 4.5.

Most widely used in fermentation product, especially ethanol, production is simultaneous saccharification and fermentation (SSF), in which there is no holding stage for the saccharification, meaning that fermenting organism(s), such as yeast, and enzyme(s), including trehalase(s), may be added together. SSF are typically carried out at temperatures from 20° C. to 40° C., such as from 26° C. to 34° C., preferably around 32° C. According to the invention the temperature may be adjusted up or down during fermentation.

In accordance with the present invention the fermentation step (iii) includes, without limitation, fermentation processes of the invention used to produce fermentation products as exemplified above in the “Fermentation Products” section.

### Processes for Producing Fermentation Products From Un-Gelatinized Starch-Containing Material

In this aspect the invention relates to processes for producing a fermentation product from starch-containing material without gelatinization (often referred to as “without cooking”) of the starch-containing material. According to the invention the desired fermentation product, such as ethanol, can be produced without liquefying the aqueous slurry containing the starch-containing material. In one embodiment a process of the invention includes saccharifying (e.g., milled) starch-containing material, e.g., granular starch, below the initial gelatinization temperature, preferably in the presence of alpha-amylase and/or carbohydrate-source generating enzyme(s) to produce sugars that can be fermented into the desired fermentation product by suitable fermenting organism(s).

In this embodiment the desired fermentation product, preferably ethanol, is produced from un-gelatinized (i.e., uncooked), preferably milled, cereal grains, such as corn.

Accordingly, in this aspect the invention relates to processes of producing a fermentation product from starch-containing material comprising the steps of:

(a) saccharifying starch-containing material at a temperature below the initial gelatinization temperature of said starch-containing material; and

(b) fermenting using one or more fermenting organisms, wherein the fermentation is carried out in the presence of one or more trehalases.

In a preferred embodiment steps (a) and (b) are carried out simultaneously (i.e., one-step fermentation).

The fermentation product, such as especially ethanol, may optionally be recovered after fermentation, e.g., by distillation. Suitable starch-containing starting materials are listed in the section “Starch-Containing Materials” section below. Contemplated enzymes are listed in the “Enzymes” section below. Typically amylase(s), such as glucoamylase(s) and/or other carbohydrate-source generating enzymes and/or alpha-amylase(s), is(are) present during fermentation.

Examples of suitable glucoamylases and other carbohydrate-source generating enzymes can be found below and includes raw starch hydrolysing glucoamylases.

Examples of suitable alpha-amylase(s) include acid alpha-amylases, preferably acid fungal alpha-amylases.

Examples of fermenting organisms include yeast, preferably a strain of Saccharomyces cerevisiae. Other suitable fermenting organisms are listed in the “Fermenting Organisms” section above.

The term “initial gelatinization temperature” means the lowest temperature at which starch gelatinization commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. In context of this invention the initial gelatinization temperature of a given starch-containing material may be determined as the temperature at which birefringence is lost in 5% of the starch granules using the method described by Gorinstein. S. and Lii. C., Starch/Stärke, Vol. 44 (12) pp. 461-466 (1992).

Before step (a) a slurry of starch-containing material, such as granular starch, having 10-55 wt. % dry solids (DS), preferably 25-45 wt. % DS, more preferably 30-40wt. % DS of starch-containing material may be prepared. The slurry may include water and/or process waters, such as stillage (backset), scrubber water, evaporator condensate or distillate, side-stripper water from distillation, or process water from other fermentation product plants. Because the process of the invention is carried out below the initial gelatinization temperature and thus no significant viscosity increase takes place, high levels of stillage may be used if desired. In an embodiment the aqueous slurry contains from about 1 to about 70 vol. %, preferably 15-60 vol. %, especially from about 30 to 50 vol. % water and/or process waters, such as stillage (backset), scrubber water, evaporator condensate or distillate, side-stripper water from distillation, or process water from other fermentation product plants, or combinations thereof, or the like.

In an embodiment backset, or other such recycled streams, subjected to trehalase treatment is added to the slurry before step (a) or during saccharification (step (a)) or simultaneous saccharification and fermentation (combined step (a) and step (b)).

The starch-containing material may be prepared by reducing the particle size, preferably by dry or wet milling, to 0.05 to 3.0 mm, preferably 0.1-0.5 mm. After being subjected to a method or process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids in the starch-containing material is converted into a soluble starch hydrolysate.

A process of the invention is conducted at a temperature below the initial gelatinization temperature, which means that the temperature at which step (a) is carried out typically lies in the range between 30-75° C., preferably between 45-60° C.

In a preferred embodiment steps (a) and (b) are carried out as a simultaneous saccharification and fermentation process. In such preferred embodiment the process is typically carried at a temperature from 20° C. to 40° C., such as from 26° C. to 34° C., preferably around 32° C.

In an embodiment fermentation is carried out so that the sugar level, such as glucose level, is kept at a low level, such as below 6 wt. %, such as below about 3 wt. %, such as below about 2 wt. %, such as below about 1 wt. %, such as below about 0.5 wt. %, or below 0.25% wt. %, such as below about 0.1 wt. %. Such low levels of sugar can be accomplished by simply employing adjusted quantities of enzyme and fermenting organism. A skilled person in the art can easily determine which doses/quantities of enzyme and fermenting organism to use. The employed quantities of enzyme and fermenting organism may also be selected to maintain low concentrations of maltose in the fermentation broth. For instance, the maltose level may be kept below about 0.5 wt. %, such as below about 0.2 wt. %.

The process of the invention may be carried out at a pH from about 3 and 7, preferably from pH 3.5 to 6, or more preferably from pH 4 to 5.

### Starch-Containing Materials

According to the processes of the invention any suitable starch-containing starting material, including granular starch (raw uncooked starch), may be used according to the present invention. The starting material is generally selected based on the desired fermentation product. Examples of starch-containing starting materials, suitable for use in methods or processes of the present invention, include tubers, roots, stems, whole grains, corns, cobs, wheat, barley, rye, milo, sago, cassava, tapioca, sorghum, rice peas, beans, or sweet potatoes, or mixtures thereof, or cereals. Contemplated are also waxy and non-waxy types of corn and barley.

The term “granular starch” means raw uncooked starch, i.e., starch in its natural form found in cereal, tubers or grains. Starch is formed within plant cells as tiny granules insoluble in water. When put in cold water, the starch granules may absorb a small amount of the liquid and swell. At temperatures up to 50° C. to 75° C. the swelling may be reversible. However, with higher temperatures an irreversible swelling called “gelatinization” begins. Granular starch to be processed may be a highly refined starch quality, preferably at least 90%, at least 95%, at least 97% or at least 99.5% pure or it may be a more crude starch-containing materials comprising (e.g., milled) whole grains including non-starch fractions such as germ residues and fibers. The raw material, such as whole grains, may be reduced in particle size, e.g., by milling, in order to open up the structure and allowing for further processing. Two processes are preferred according to the invention: wet and dry milling. In dry milling whole kernels are milled and used. Wet milling gives a good separation of germ and meal (starch granules and protein) and is often applied at locations where the starch hydrolysate is used in production of, e.g., syrups. Both dry and wet milling is well known in the art of starch processing and is equally contemplated for a process of the invention. In an embodiment the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen.

Production of Fermentation Products from Lignocellulose-Containing Material

In this aspect, the invention relates to processes of producing fermentation products from lignocellulose-containing material. Conversion of lignocellulose-containing material into fermentation products, such as ethanol, has the advantages of the ready availability of large amounts of feedstock, including wood, agricultural residues, herbaceous crops, municipal solid wastes etc. Lignocellulose-containing materials typically primarily consist of cellulose, hemicellulose, and lignin and are often referred to as “biomass”.

The structure of lignocellulose is not directly accessible to enzymatic hydrolysis. Therefore, the lignocellulose-containing material has to be pre-treated, e.g., by acid hydrolysis under adequate conditions of pressure and temperature, in order to break the lignin seal and disrupt the crystalline structure of cellulose. This causes solubilization of the hemicellulose and cellulose fractions. The cellulose and hemicelluloses can then be hydrolyzed enzymatically, e.g., by cellulolytic and/or hemicellulolytic enzymes, to convert the carbohydrate polymers into fermentable sugars which may be fermented into desired fermentation products, such as ethanol. Optionally the fermentation product may be recovered, e.g., by distillation as also described above.

In this aspect the invention relates to processes of producing a fermentation product from lignocellulose-containing material, comprising the steps of:

(a) pre-treating lignocellulose-containing material;
(b) hydrolyzing the material;
(c) fermenting with one or more fermenting organisms in the presence of one or more trehalases.

The trehalase(s) may be added before and/or during fermentation. In an embodiment trehalase treatment is carried out after pre-treatment, such as during hydrolysis in step (b).

Hydrolysis steps (b) and fermentation step (c) may be carried out sequentially or simultaneously. In preferred embodiments the steps are carried out as SSF, HHF or SHF process steps which will be described further below.

### SSF, HHF and SHF

In one embodiment of the present invention, hydrolysis and fermentation is carried out as a simultaneous hydrolysis and fermentation step (SSF). In general this means that combined/simultaneous hydrolysis and fermentation are carried out at conditions (e.g., temperature and/or pH) suitable, preferably optimal, for the fermenting organism(s) in question.

In another embodiment hydrolysis step and fermentation step are carried out as hybrid hydrolysis and fermentation (HHF). HHF typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable, preferably optimal, for the hydrolyzing enzyme(s) in question. The subsequent simultaneous hydrolysis and fermentation step is typically carried out at conditions suitable for the fermenting organism(s) (often at lower temperatures than the separate hydrolysis step).

In another embodiment, the hydrolysis and fermentation steps may also be carried out as separate hydrolysis and fermentation, where the hydrolysis is taken to completion before initiation of fermentation. This is often referred to as “SHF”.

### Pre-Treatment

The lignocellulose-containing material may according to the invention be pre-treated before being hydrolyzed and fermented. In a preferred embodiment the pre-treated material is hydrolyzed, preferably enzymatically, before and/or during fermentation. The goal of pre-treatment is to separate and/or release cellulose, hemicellulose and/or lignin and this way improve the rate of enzymatic hydrolysis.

According to the invention pre-treatment step (a) may be a conventional pre-treatment step known in the art. Pre-treatment may take place in aqueous slurry. The lignocellulose-containing material may during pre-treatment be present in an amount between 10-80 wt. %, preferably between 20-50 wt. %.

Chemical, Mechanical and/or Biological Pre-Treatment

The lignocellulose-containing material may according to the invention be chemically, mechanically and/or biologically pre-treated before hydrolysis and/or fermentation. Mechanical treatment (often referred to as physical pre-treatment) may be used alone or in combination with subsequent or simultaneous hydrolysis, especially enzymatic hydrolysis, to promote the separation and/or release of cellulose, hemicellulose and/or lignin.

Preferably, the chemical, mechanical and/or biological pre-treatment is carried out prior to the hydrolysis and/or fermentation. Alternatively, the chemical, mechanical and/or biological pre-treatment is carried out simultaneously with hydrolysis, such as simultaneously with addition of one or more cellulolytic enzymes, or other enzyme activities mentioned below, to release fermentable sugars, such as glucose and/or maltose.

In an embodiment of the invention the pre-treated lignocellulose-containing material is washed and/or detoxified before or after hydrolysis step (b). This may improve the fermentability of, e.g., dilute-acid hydrolyzed lignocellulose-containing material, such as corn stover. Detoxification may be carried out in any suitable way, e.g., by steam stripping, evaporation, ion exchange, resin or charcoal treatment of the liquid fraction or by washing the pre-treated material.

### Chemical Pre-Treatment

According to the present invention “chemical pre-treatment” refers to any chemical treatment which promotes the separation and/or release of cellulose, hemicellulose and/or lignin. Examples of suitable chemical pre-treatment steps include treatment with; for example, dilute acid, lime, alkaline, organic solvent, ammonia, sulphur dioxide, carbon dioxide. Further, wet oxidation and pH-controlled hydrothermolysis are also contemplated chemical pre-treatments.

Preferably, the chemical pre-treatment is acid treatment, more preferably, a continuous dilute and/or mild acid treatment, such as, treatment with sulfuric acid, or another organic acid, such as acetic acid, citric acid, tartaric acid, succinic acid, or mixtures thereof. Other acids may also be used. Mild acid treatment means in the context of the present invention that the treatment pH lies in the range from 1-5, preferably from pH 1-3. In a specific embodiment the acid concentration is in the range from 0.1 to 2.0 wt. % acid, preferably sulphuric acid. The acid may be mixed or contacted with the material to be fermented according to the invention and the mixture may be held at a temperature in the range of 160-220° C., such as 165-195° C., for periods ranging from minutes to seconds, e.g., 1-60 minutes, such as 2-30 minutes or 3-12 minutes. Addition of strong acids, such as sulphuric acid, may be applied to remove hemicellulose. This enhances the digestibility of cellulose.

Cellulose solvent treatment, also contemplated according to the invention, has been shown to convert about 90% of cellulose to glucose. It has also been shown that enzymatic hydrolysis could be greatly enhanced when the lignocellulosic structure is disrupted. Alkaline H₂O₂, ozone, organosolv (uses Lewis acids, FeCl₃, (Al)₂SO₄ in aqueous alcohols), glycerol, dioxane, phenol, or ethylene glycol are among solvents known to disrupt cellulose structure and promote hydrolysis (Mosier et al. Bioresource Technology 96 (2005), p. 673-686).

Alkaline chemical pre-treatment with base, e.g., NaOH, Na₂CO₃ and/or ammonia or the like, is also within the scope of the invention. Pre-treatment methods using ammonia are described in, e.g., WO 2006/110891, WO 2006/11899, WO 2006/11900, WO 2006/110901, which are hereby incorporated by reference to the extent that they teach alkaline chemical pre-treatment.

Wet oxidation techniques involve use of oxidizing agents, such as: sulphite based oxidizing agents or the like. Examples of solvent pre-treatments include treatment with DMSO (Dimethyl Sulfoxide) or the like. Chemical pre-treatment is generally carried out for 1 to 60 minutes, such as from 5 to 30 minutes, but may be carried out for shorter or longer periods of time dependent on the material to be pre-treated.

Other examples of suitable pre-treatment methods are described by Schell et al. (2003) Appl. Biochem and Biotechn. Vol. 105-108, p. 69-85, and Mosier et al. Bioresource Technology 96 (2005) 673-686, and US publication no. 2002/0164730, which references are hereby all incorporated by reference to the extent that they teach pre-treatment methods.

### Mechanical Pre-Treatment

As used in context of the present invention the term “mechanical pre-treatment” refers to any mechanical or physical pre-treatment which promotes the separation and/or release of cellulose, hemicellulose and/or lignin from lignocellulose-containing material. For example, mechanical pre-treatment includes various types of milling, irradiation, steaming/steam explosion, and hydrothermolysis.

Mechanical pre-treatment includes comminution (mechanical reduction of the particle size). Comminution includes dry milling, wet milling and vibratory ball milling. Mechanical pre-treatment may involve high pressure and/or high temperature (steam explosion). In an embodiment of the invention high pressure means pressure in the range from 300 to 600 psi, preferably 400 to 500 psi, such as around 450 psi. In an embodiment of the invention high temperature means temperatures in the range from about 100 to 300° C., preferably from about 140 to 235° C. In a preferred embodiment mechanical pre-treatment is a batch-process, steam gun hydrolyzer system which uses high pressure and high temperature as defined above. A Sunds Hydrolyzer (available from Sunds Defibrator AB (Sweden) may be used for this.

### Combined Chemical and Mechanical Pre-Treatment

In an embodiment of the invention both chemical and mechanical pre-treatments are carried out involving, for example, both dilute or mild acid pretreatment and high temperature and pressure treatment. The chemical and mechanical pretreatment may be carried out sequentially or simultaneously, as desired.

Accordingly, in a preferred embodiment, the lignocellulose-containing material is subjected to both chemical and mechanical pre-treatment to promote the separation and/or release of cellulose, hemicellulose and/or lignin.

In a preferred embodiment the pre-treatment is carried out as a dilute and/or mild acid steam explosion step. In another preferred embodiment pre-treatment is carried out as an ammonia fiber explosion step or AFEX pretreatment step

### Biological Pre-Treatment

As used in the present invention the term “biological pre-treatment” refers to any biological pre-treatment which promotes the separation and/or release of cellulose, hemicellulose, and/or lignin from the lignocellulose-containing material. Biological pre-treatment techniques can involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, DC, 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, DC, chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

### Hydrolysis

Before and/or during fermentation the pre-treated lignocellulose-containing material may be hydrolyzed in order to break the lignin seal and disrupt the crystalline structure of cellulose. In a preferred embodiment hydrolysis is carried out enzymatically. According to the invention the pre-treated lignocellulose-containing material to be fermented may be hydrolyzed by one or more hydrolases (class E.C. 3 according to Enzyme Nomenclature), preferably one or more carbohydrases including cellulolytic enzymes and hemicellulolytic enzymes, or combinations thereof. Further, protease, alpha-amylase, glucoamylase and/or the like may also be present during hydrolysis and/or fermentation as the lignocellulose-containing material may include some, e.g., starchy and/or proteinaceous material.

The enzyme(s) used for hydrolysis may be capable of directly or indirectly converting carbohydrate polymers into fermentable sugars, such as glucose and/or maltose, which can be fermented into a desired fermentation product, such as ethanol.

In a preferred embodiment the carbohydrase(s) has(have) cellulolytic and/or hemicellulolytic enzyme activity.

In a preferred embodiment hydrolysis is carried out using a cellulolytic enzyme preparation further comprising one or more polypeptides having cellulolytic enhancing activity.

In a preferred embodiment the polypeptide(s) having cellulolytic enhancing activity is(are) of family GH61A origin. Examples of suitable and preferred cellulolytic enzyme preparations and polypeptides having cellulolytic enhancing activity are described in the “Cellulolytic Enzymes” section and “Cellulolytic Enhancing Polypeptides” sections below.

Suitable enzymes are described in the “Enzymes” section below.

Hemicellulose polymers can be broken down by hemicellullolytic enzymes and/or acid hydrolysis to release its five and six carbon sugar components. The six carbon sugars (hexoses), such as glucose, galactose, arabinose, and mannose, can readily be fermented to fermentation products such as ethanol, acetone, butanol, glycerol, citric acid, fumaric acid etc. by suitable fermenting organisms, including yeast.

Enzymatic hydrolysis is preferably carried out in a suitable aqueous environment under conditions which can readily be determined by one skilled in the art. In a preferred embodiment hydrolysis is carried out at suitable, preferably optimal, conditions for the enzyme(s) in question.

Suitable process time, temperature and pH conditions can readily be determined by one skilled in the art. Preferably, hydrolysis is carried out at a temperature between 25 and 70° C., preferably between 40 and 60° C., especially around 50° C. The step is preferably carried out at a pH in the range from 3-8, preferably pH 4-6. Hydrolysis is typically carried out for between 12 and 96 hours, preferable 16 to 72 hours, more preferably between 24 and 48 hours.

Fermentation of lignocellulose derived material is carried out in accordance with a fermentation method of the invention as described above.

### Lignocellulose-Containing Material (Biomass)

Any suitable lignocellulose-containing material is contemplated in context of the present invention. Lignocellulose-containing material may be any material containing lignocellulose. In a preferred embodiment the lignocellulose-containing material contains at least 50 wt. %, preferably at least 70 wt. %, more preferably at least 90 wt. % lignocellulose. It is to be understood that the lignocellulose-containing material may also comprise other constituents such as cellulosic material, such as cellulose, hemicellulose and may also comprise constituents such as sugars, such as fermentable sugars and/or un-fermentable sugars.

Lignocellulose-containing material is generally found, for example, in the stems, leaves, hulls, husks, and cobs of plants or leaves, branches, and wood of trees. Lignocellulosic material can also be, but is not limited to, herbaceous material, agricultural residues, forestry residues, municipal solid wastes, waste paper, and pulp and paper mill residues. It is understood herein that lignocellulose-containing material may be in the form of plant cell wall material containing lignin, cellulose, and hemi-cellulose in a mixed matrix.

In an embodiment the lignocellulose-containing material is corn fiber, rice straw, pine wood, wood chips, bagasse, paper and pulp processing waste, corn stover, corn cobs, hardwood such as poplar and birch, softwood, cereal straw such as wheat straw, switch grass, miscanthus, rice hulls, municipal solid waste (MSW), industrial organic waste, office paper, or mixtures thereof.

In a preferred embodiment the lignocellulose-containing material is corn stover or corn cobs. In another preferred embodiment, the lignocellulose-containing material is corn fiber. In another preferred embodiment, the lignocellulose-containing material is switch grass. In another preferred embodiment, the the lignocellulose-containing material is bagasse.

### High Pitch Fermentation

In another embodiment the invention relates to processes of producing fermentation products from lignocellulose-containing material, wherein the method comprises:

a) pre-treating lignocellulose-containing material;

b) hydrolysing the pre-treated lignocellulose-containing material;

c) fermenting using one or more fermenting organisms;

wherein fermentation is initiated and carried out at:

i) a fermentation organism cell count in the range from 10-250×10¹⁰ cells per L fermentation medium; or

ii) a fermentation organism concentration in the range from 2-50 g TS fermenting organism per L fermentation medium,

wherein one or more trehalases are present in the fermentation medium.

At high yeast concentrations the amount of trehalose which can be converted to a desired fermentation product is high. In a preferred embodiment insoluble solids (including lignin and unconverted polysaccharides) are removed before and/or during fermentation. For instance, the insoluble solids may be removed after pre-treating the lignocellulose-containing material in step a). The pre-treated lignocellulose derived material, having insoluble solids removed, may then be fermented in accordance with the invention. In another embodiment the insoluble solids may be removed after hydrolyzing the pre-treated lignocellulose-containing material in step b). The hydrolyzed pre-treated lignocellulose derived material, having insoluble solids removed, may then be fermented in accordance with the invention.

The lignocellulose derived fermentable sugars to be fermented are in the form of liquor (e.g., filtrate) coming from the pre-treatment and/or hydrolysis steps a) and/or b). In a preferred embodiment hydrolysis in step b) and fermentation in step c) are carried out as a hybrid hydrolysis and fermentation step (HHF), as a simultaneous hydrolysis and fermentation (SSF) or separate hydrolysis and fermentation. SSF, HHF and SHF are well known steps in the art.

In a preferred embodiment fermentation may be carried out at a fermentation organism cell count in the range from between 20-250×10¹⁰ cells per L fermentation medium, more preferably in the range from 50-250×10¹⁰ cells per L fermentation medium, more preferably in the range from 100-250×10¹⁰ cells per L fermentation medium, more preferably in the range from 150-250×10¹⁰ cells per L fermentation medium, such as in the range from 200-250×10¹⁰ cells per L fermentation medium.

In a preferred embodiment fermentation may be carried out at a fermentation organism concentration in the range from 3-50 g TS (total solids) fermenting organism per L fermentation medium, preferably in the range from 4-50 g TS fermenting organism per L fermentation medium, preferably in the range from 5-50 g TS fermenting organism per L fermentation medium, more preferably in the range from 10-50 g TS fermenting organism per L fermentation medium, more preferably in the range from 20-50 g TS fermenting organism per L fermentation medium; especially in the range from 30-50 g TS fermenting organism per L fermentation medium.

According to the invention the fermenting organisms may be immobilized. For instance, the fermenting organisms may be immobilized on inert, high surface area supports which are suspended in the fermentation tank/vessel through which hydrolysed and/or pre-treated lignocellulose derived material to be fermented is fed. Any immobilization technique may be used according to the invention. Techniques for immobilizing fermenting organisms are well known in the art. Examples of suitable immobilizing techniques can be found in, e.g., Kesava et al., (1996), “Ethanol production by immobilized whole cells of Zymomonas mobilis in a continuous flow expanded bed bioreactor and a continuous flow stirred tank bioreactor”, Journal of Industrial Microbiology 17:11-14; Gough S et al., (1998), “Production of ethanol from molasses at 45 degrees C using Kluyveromyces marxianus IMB3 immobilized in calcium alginate gels and poly(vinyl alcohol) cryogel”, Bioprocess Engineering 19:87-90; Love et al., (1998), “Continuous ethanol fermentation at 45 degrees C. using Kluyveromyces marxianus IMB3 immobilized in Calcium alginate and kissiris”, Bioprocess Engineering 18:187-189; Abbi et al., (1996), “Bioconversion of pentose sugars to ethanol by free and immobilized cells of Candida shehatae (NCL-3501): Fermentation behaviour” Process Biochemistry 31:555-560; Krishnan et al., (2000), “Ethanol production from glucose and xylose by immobilized Zymomonas mobilis CP4(pZB5)”, Applied Biochemistry And Biotechnology 84-6:525-541; Chibata et al. (1981) Ann. Rev. Microphys. Bioeng 10: 197-216; Fukui et al (1982) Ann. Rev. Microbial 36: 145-172; John F. Kennedy, (1982) Nature, 299: 777-778 (all refs are hereby incorporated by reference).

The fermenting organisms may advantageously be recovered and re-used. For instance, the fermenting organisms may be recovered by separating them from the fermentation medium in the fermentation tank/vessel. Alternatively, the fermenting organisms may be recovered by separating them from the fermentation medium after fermentation. The fraction of the fermentation medium that contains the fermentation product may be further processed or recovered, e.g., by distillation. The recovered fermentation organisms may be recycled to the same fermentation tank/vessel or to one or more other fermentation tanks/vessels. In other words, the fermenting organisms may be recovery and recycled to the fermentation medium and this way re-used in one or more additional fermentation cycles in accordance with the invention.

Any technique may be used for recovering the fermenting organisms. Suitable techniques well known in the art include filtration, e.g., using a filter press, and centrifugation.

### Lyzing Fermenting Organisms

Trehalose is produced by fermenting organisms, such as yeast, as a response to stress. This trehalose may according to the invention be hydrolyzed into glucose and used in a fermentation process for producing a fermentation product, such as ethanol.

According to this aspect the invention relates to processes of producing a fermentation product comprising:

i) fermenting plant material derived sugars using one or more fermenting organisms;
ii) treating the fermenting plant material with one or more trehalase;
ii) recycling trehalase treated material to step i).

The plant material may in one embodiment be starch-containing material. The plant material may in another embodiment be lignocellulose-containing material.

The sugars may be derived from starch-containing material which has been subjected to a conventional starch-to-sugar process of the type described in the section “Processes for producing fermentation products from gelatinized starch-containing material,” comprising the steps of liquefying starch-containing material and saccharifying the liquefied material. The liquefied and optionally saccharified material may be used in fermentation step i). According to the invention saccharification, in order to provide fermentable sugars, may take place simultaneously with fermentation in step i), i.e., SSF process. In an embodiment the plant material in step i) is gelatinized starch-containing material which has been subjected to a liquefaction step and optionally saccharification.

In another embodiment the sugars are derived from starch-containing material which has been subjected to a raw starch hydrolysis process of the type described in the section “Processes for producing fermentation products from un-gelatinized starch-containing material,” wherein step i) is carried out by simultaneously saccharifying and fermenting un-gelatinized starch material in step i). In other words, step i) may be carried out as a one-step fermentation of ungelatinized starch material (or uncooked starch) below the initial gelatinization temperature. In an embodiment the plant material in step i) is un-gelatinized starch-containing material which has optionally been subjected to saccharification.

In another embodiment the sugars are derived from lignocellulose-containing material using a process of the type described in the section “Production of Fermentation Products from Lignocellulose-Containing Material,” comprising pre-treating lignocellulose-containing material; and optionally hydrolyzing the material. Fermentation in step i) may, e.g., be carried out as a SSF, HHF or SHF.

In an embodiment the fermenting organism(s) in the fermentation medium in step i) is subjected to one or more fermenting organism lyzing enzymes including, without being limited to, one or more enzyme selected from the group of proteases, pectinases, beta-glucanases, chitinases, or mixtures comprising one or more enzymes thereof.

In an embodiment the fermenting organism(s) used for fermentation in step i) is separated from the fermentation medium before subjecting it to trehalase treatment in step ii).

The trehalose obtained after lyzing of the fermenting organism(s) may be subjected to trehalase before introduction into fermentation step i).

Recycling may be done in any suitable way. One skilled in the art would know how to recycle the trehalase treated material.

### Enzymes

Even if not specifically mentioned in context of methods and/or processes of the invention, it is to be understood that enzyme(s) is(are) used in effective amounts.

### Alpha-Amylase

According to the invention any alpha-amylase may be used, such as of fungal, bacterial or plant origin. In a preferred embodiment the alpha-amylase is an acid alpha-amylase, e.g., acid fungal alpha-amylase or acid bacterial alpha-amylase. The term “acid alpha-amylase” means an alpha-amylase (E.C. 3.2.1.1) which added in an effective amount has activity optimum at a pH in the range of 3 to 7, preferably from 3.5 to 6, or more preferably from 4-5.

### Bacterial Alpha-Amylase

According to the invention a bacterial alpha-amylase is preferably derived from the genus Bacillus.

In a preferred embodiment the Bacillus alpha-amylase is derived from a strain of Bacillus licheniformis, Bacillus amyloliquefaciens, Bacillus subtilis or Bacillus stearothermophilus, but may also be derived from other Bacillus sp. Specific examples of contemplated alpha-amylases include the Bacillus licheniformis alpha-amylase shown in SEQ ID NO: 4 in WO 99/19467, the Bacillus amyloliquefaciens alpha-amylase SEQ ID NO: 5 in WO 99/19467 and the Bacillus stearothermophilus alpha-amylase shown in SEQ ID NO: 3 in WO 99/19467 (all sequences hereby incorporated by reference). In an embodiment the alpha-amylase may be an enzyme having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 1, 2 or 3, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 or U.S. Pat. No. 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873—see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted I181*+G182*+N193F) compared to the wild-type BSG alpha-amylase amino acid sequence set forth in SEQ ID NO:3 disclosed in WO 99/19467.

### Bacterial Hybrid Alpha-Amylase

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitution:

G48A+T49I+G107A+H156Y+A181T+N190F+1201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

In an embodiment the bacterial alpha-amylase is dosed in an amount of 0.0005-5 KNU per g DS, preferably 0.001-1 KNU per g DS, such as around 0.050 KNU per g DS.

### Fungal Alpha-Amylase

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae. According to the present invention, the term “Fungamyl-like alpha-amylase” indicates an alpha-amylase which exhibits a high identity, i.e. at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes NS, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al. J. Ferment. Bioeng 81:292-298(1996) “Molecular-cloning and determination of the nucleotide-sequence of a gene encoding an acid-stable alpha-amylase from Aspergillus kawachii”; and further as EMBL: #AB008370.

The fungal alpha-amylase may also be a wild-type enzyme comprising a starch-binding domain (SBD) and an alpha-amylase catalytic domain (i.e., none-hybrid), or a variant thereof. In an embodiment the wild-type alpha-amylase is derived from a strain of Aspergillus kawachii.

### Fungal Hybrid Alpha-Amylase

In a preferred embodiment the fungal acid alpha-amylase is a hybrid alpha-amylase. Preferred examples of fungal hybrid alpha-amylases include the ones disclosed in WO 2005/003311 or U.S. Patent Publication no. 2005/0054071 (Novozymes) or US patent application no. WO 2006/069290 (Novozymes) which is hereby incorporated by reference. A hybrid alpha-amylase may comprise an alpha-amylase catalytic domain (CD) and a carbohydrate-binding domain/module (CBM), such as a starch binding domain, and optional a linker.

Specific examples of contemplated hybrid alpha-amylases include those disclosed in Table 1 to 5 of the examples in WO 2006/069290, including Fungamyl variant with catalytic domain JA118 and Athelia rolfsii SBD (SEQ ID NO:100 in WO 2006/069290), Rhizomucor pusillus alpha-amylase with Athelia rolfsii AMG linker and SBD (SEQ ID NO:101 in US WO 2006/069290), Rhizomucor pusillus alpha-amylase with Aspergillus niger glucoamylase linker and SBD (which is disclosed in Table 5 as a combination of amino acid sequences SEQ ID NO:20, SEQ ID NO:72 and SEQ ID NO:96 in U.S. application Ser. No. 11/316,535) or as V039 in Table 5 in WO 2006/069290, and Meripilus giganteus alpha-amylase with Athelia rolfsii glucoamylase linker and SBD (SEQ ID NO:102 in US WO 2006/069290). Other specifically contemplated hybrid alpha-amylases are any of the ones listed in Tables 3, 4, 5, and 6 in Example 4 in U.S. application Ser. No. 11/316,535 and WO 2006/069290 (hereby incorporated by reference)

Other specific examples of contemplated hybrid alpha-amylases include those disclosed in U.S. Patent Publication no. 2005/0054071, including those disclosed in Table 3 on page 15, such as Aspergillus niger alpha-amylase with Aspergillus kawachii linker and starch binding domain.

Contemplated are also alpha-amylases which exhibit a high identity to any of above mention alpha-amylases, i.e., at least 70%, at least 75%, at least 80%, at least 85%, at least 90%, at least 95%, at least 96%, at least 97%, at least 98%, at least 99% or even 100% identity to the mature enzyme sequences.

An acid alpha-amylases may according to the invention be added in an amount of 0.001 to 10 AFAU/g DS, preferably from 0.01 to 5 AFAU/g DS, especially 0.3 to 2 AFAU/g DS or 0.001 to 1 FAU-F/g DS, preferably 0.01 to 1 FAU-F/g DS.

### Commercial Alpha-Amylase Products

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYME™ SC and SAN™ SUPER, SAN™ EXTRA L (Novozymes NS) and CLARASE™ L-40,000, DEX-LO™, SPEZYME™ FRED, SPEZYME™ AA, and SPEZYME™ DELTA AA (Genencor Int.), and the acid fungal alpha-amylase sold under the trade name SP288 (available from Novozymes NS, Denmark).

### Carbohydrate-Source Generating Enzyme

The term “carbohydrate-source generating enzyme” includes glucoamylase (being glucose generators), beta-amylase and maltogenic amylase (being maltose generators) and also pullulanase and alpha-glucosidase. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated blends are mixtures comprising at least a glucoamylase and an alpha-amylase, especially an acid amylase, even more preferred an acid fungal alpha-amylase. The ratio between glucoamylase activity (AGU) and acid fungal alpha-amylase activity (FAU-F) (i.e., AGU per FAU-F) may in a preferred embodiment of the invention be between 0.1 and 100 AGU/FAU-F, in particular between 2 and 50 AGU/FAU-F, such as in the range from 10-40 AGU/FAU-F, especially when doing one-step fermentation (Raw Starch Hydrolysis—RSH), i.e., when saccharification in step (a) and fermentation in step (b) are carried out simultaneously (i.e. without a liquefaction step).

In a conventional starch-to-ethanol process (i.e., including a liquefaction step (a)) the ratio may preferably be as defined in EP 140,410-B1, especially when saccharification in step ii) and fermentation in step iii) are carried out simultaneously.

### Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka, Y. et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulata, Pachykytospora papyracea; and Leucopaxillus giganteus all disclosed in WO 2006/069289; or Peniophora rufomarginata disclosed in PCT/US2007/066618; or a mixture thereof. Also hybrid glucoamylase are contemplated according to the invention. Examples the hybrid glucoamylases disclosed in WO 2005/045018. Specific examples include the hybrid glucoamylase disclosed in Table 1 and 4 of Example 1 (which hybrids are hereby incorporated by reference).

Contemplated are also glucoamylases which exhibit a high identity to any of above mention glucoamylases, i.e., at least 70%, at least 75%, at least 80%, at least 85%, at least 90%, at least 95%, at least 96%, at least 97%, at least 98%, at least 99% or even 100% identity to the mature enzymes sequences mentioned above.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME ULTRA™ and AMG™ E (from Novozymes NS, Denmark); OPTIDEX™ 300, GC480™ and GC147™ (from Genencor Int., USA); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, especially between 1-5 AGU/g DS, such as 0.1-2 AGU/g DS, such as 0.5 AGU/g DS or in an amount of 0.0001-20 AGU/g DS, preferably 0.001-10 AGU/g DS, especially between 0.01-5 AGU/g DS, such as 0.1-2 AGU/g DS.

### Beta-Amylase

A beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is NOVOZYM™ WBA from Novozymes NS, Denmark and SPEZYME™ BBA 1500 from Genencor Int., USA.

### Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Cellulolytic Activity

The term “cellulolytic activity” as used herein are understood as comprising enzymes having cellobiohydrolase activity (EC 3.2.1.91), e.g., cellobiohydrolase I and cellobiohydrolase II, as well as endo-glucanase activity (EC 3.2.1.4) and beta-glucosidase activity (EC 3.2.1.21).

In order to be efficient, the digestion of cellulose may require several types of enzymes acting cooperatively. At least three categories of enzymes are often needed to convert cellulose into glucose: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

The cellulases may comprise a carbohydrate-binding module (CBM) which enhances the binding of the enzyme to a cellulose-containing fiber and increases the efficacy of the catalytic active part of the enzyme. A CBM is defined as contiguous amino acid sequence within a carbohydrate-active enzyme with a discreet fold having carbohydrate-binding activity. For further information of CBMs see the CAZy internet server (Supra) or Tomme et al. (1995) in Enzymatic Degradation of Insoluble Polysaccharides (Saddler and Penner, eds.), Cellulose-binding domains: classification and properties. pp. 142-163, American Chemical Society, Washington.

The cellulolytic activity may, in a preferred embodiment, be in the form of a preparation of enzymes of fungal origin, such as from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense (see e.g., US publication #2007/0238155 from Dyadic Inc, USA).

In preferred embodiment the cellulolytic enzyme preparation contains one or more of the following activities: cellulase, hemicellulase, cellulolytic enzyme enhancing activity, beta-glucosidase activity, endoglucanase, cellubiohydrolase, or xylose-isomerase.

In a preferred embodiment the cellulases or cellulolytic enzymes may be a cellulolytic preparation as defined PCT/2008/065417, which is hereby incorporated by reference. In a preferred embodiment the cellulolytic preparation comprising a polypeptide having cellulolytic enhancing activity (GH61A), preferably the one disclosed in WO 2005/074656. The cellulolytic preparation may further comprise a beta-glucosidase, such as a beta-glucosidase derived from a strain of the genus Trichoderma, Aspergillus or Penicillium, including the fusion protein having beta-glucosidase activity disclosed in WO2008/057637 (Novozymes). In an embodiment the cellulolytic preparation may also comprises a CBH II, preferably Thielavia terrestris cellobiohydrolase II (CEL6A). In another preferred embodiment the cellulolytic enzyme preparation may also comprise cellulolytic enzymes, preferably one derived from Trichoderma reesei, Humicola insolens and/or Chrysosporium lucknowense.

In an embodiment the cellulolytic enzyme preparation comprises a polypeptide having cellulolytic enhancing activity (GH61A) disclosed in WO 2005/074656; a cellobiohydrolase, such as Thielavia terrestris cellobiohydrolase II (CEL6A), a beta-glucosidase (e.g., the fusion protein disclosed in WO2008/057634) and cellulolytic enzymes, e.g., derived from Trichoderma reesei.

In an embodiment the cellulolytic enzyme preparation comprises a polypeptide having cellulolytic enhancing activity (GH61A) disclosed in WO 2005/074656; a beta-glucosidase (e.g., the fusion protein disclosed in WO 2008/057637) and cellulolytic enzymes, e.g., derived from Trichoderma reesei.

In an embodiment the cellulolytic enzyme composition is the commercially available product CELLUCLAST™ 1.5L, CELLUZYME™ (from Novozymes NS, Denmark) or ACCELERASE™ 1000 (from Genencor Inc. USA).

A cellulase may be added for hydrolyzing the pre-treated lignocellulose-containing material. The cellulase may be dosed in the range from 0.1-100 FPU per gram total solids (TS), preferably 0.5-50 FPU per gram TS, especially 1-20 FPU per gram TS. In another embodiment at least 0.1 mg cellulolytic enzyme per gram total solids (TS), preferably at least 3 mg cellulolytic enzyme per gram TS, such as between 5 and 10 mg cellulolytic enzyme(s) per gram TS is(are) used for hydrolysis.

### Endoglucanase (EG)

The term “endoglucanase” means an endo-1,4-(1,3;1,4)-beta-D-glucan 4-glucanohydrolase (E.C. No. 3.2.1.4), which catalyses endo-hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity may be determined using carboxymethyl cellulose (CMC) hydrolysis according to the procedure of Ghose, 1987, Pure and Appl. Chem. 59: 257-268.

In a preferred embodiment endoglucanases may be derived from a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

### Cellobiohydrolase (CBH)

The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydroloses are mentioned above including CBH I and CBH II from Trichoderma reseei; Humicola insolens and CBH II from Thielavia terrestris cellobiohydrolase

### (CELL6A)

Cellobiohydrolase activity may be determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288. The Lever et al. method is suitable for assessing hydrolysis of cellulose in corn stover and the method of van Tilbeurgh et al. is suitable for determining the cellobiohydrolase activity on a fluorescent disaccharide derivative.

### Beta-Glucosidase

One or more beta-glucosidases may be present during hydrolysis.

The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42: 55-66, except different conditions were employed as described herein. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

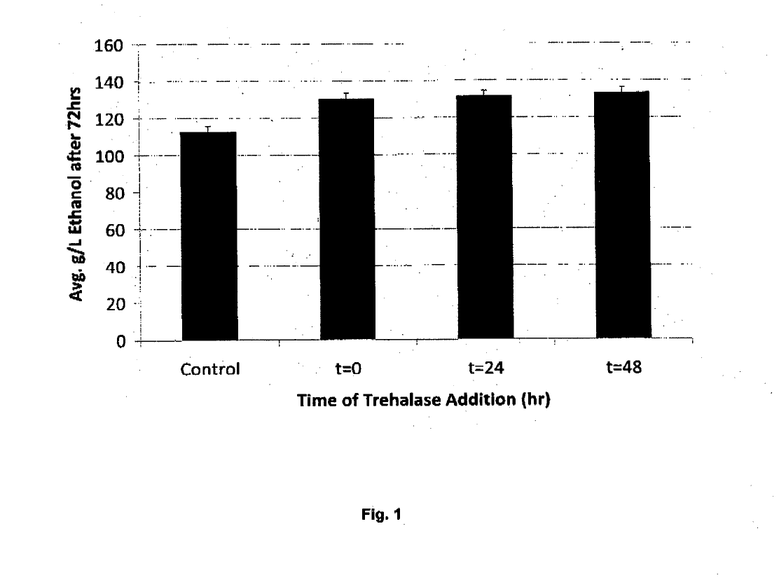

In a preferred embodiment the beta-glucosidase is of fungal origin, such as a strain of the genus Trichoderma, Aspergillus or Penicillium. In a preferred embodiment the beta-glucosidase is a derived from Trichoderma reesei, such as the beta-glucosidase encoded by the bgl1 gene (see FIG. 1 of EP 562003). In another preferred embodiment the beta-glucosidase is derived from Aspergillus oryzae (recombinantly produced in Aspergillus oryzae according to WO 02/095014), Aspergillus fumigatus (recombinantly produced in Aspergillus oryzae according to Example 22 of WO 02/095014) or Aspergillus niger (1981, J. Appl. Vol 3, pp 157-163).

### Hemicellulolytic Enzymes

According to the invention the pre-treated lignocellulose-containing material may further be subjected to one or more hemicellulolytic enzymes, e.g., one or more hemicellulases.

Hemicellulose can be broken down by hemicellulases and/or acid hydrolysis to release its five and six carbon sugar components.

In an embodiment of the invention the lignocellulose derived material may be treated with one or more hemicellulases.

Any hemicellulase suitable for use in hydrolyzing hemicellulose, preferably into xylose, may be used. Preferred hemicellulases include xylanases, arabinofuranosidases, acetyl xylan esterase, feruloyl esterase, glucuronidases, endo-galactanase, mannases, endo or exo arabinases, exo-galactanses, and mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes NS, Denmark).

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes NS, Denmark.

The hemicellulase may be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

### Cellulolytic Enhancing Activity

The term “cellulolytic enhancing activity” is defined herein as a biological activity that enhances the hydrolysis of a lignocellulose derived material by proteins having cellulolytic activity. For purposes of the present invention, cellulolytic enhancing activity is determined by measuring the increase in reducing sugars or in the increase of the total of cellobiose and glucose from the hydrolysis of a lignocellulose derived material, e.g., pre-treated lignocellulose-containing material by cellulolytic protein under the following conditions: 1-50 mg of total protein/g of cellulose in PCS (pre-treated corn stover), wherein total protein is comprised of 80-99.5% w/w cellulolytic protein/g of cellulose in PCS and 0.5-20% w/w protein of cellulolytic enhancing activity for 1-7 day at 50° C. compared to a control hydrolysis with equal total protein loading without cellulolytic enhancing activity (1-50 mg of cellulolytic protein/g of cellulose in PCS).

The polypeptides having cellulolytic enhancing activity enhance the hydrolysis of a lignocellulose derived material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

In a preferred embodiment the hydrolysis and/or fermentation is carried out in the presence of a cellulolytic enzyme in combination with a polypeptide having enhancing activity. In a preferred embodiment the polypeptide having enhancing activity is a family GH61A polypeptide. WO 2005/074647 discloses isolated polypeptides having cellulolytic enhancing activity and polynucleotides thereof from Thielavia terrestris. WO 2005/074656 discloses an isolated polypeptide having cellulolytic enhancing activity and a polynucleotide thereof from Thermoascus aurantiacus. U.S. Published Application Serial No. 2007/0077630 discloses an isolated polypeptide having cellulolytic enhancing activity and a polynucleotide thereof from Trichoderma reesei.

### Xylose Isomerase

Xylose isomerases (D-xylose ketoisomerase) (E.C. 5.3.1.5.) are enzymes that catalyze the reversible isomerization reaction of D-xylose to D-xylulose. Some xylose isomerases also convert the reversible isomerization of D-glucose to D-fructose. Therefore, xylose isomarase is sometimes referred to as “glucose isomerase.”

A xylose isomerase used in a method or process of the invention may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus, Flavobacterium, and Thermotoga, including T. neapolitana (Vieille et al., 1995, Appl. Environ. Microbiol. 61(5): 1867-1875) and T. maritime.

Examples of fungal xylose isomerases are derived species of Basidiomycetes.

A preferred xylose isomerase is derived from a strain of yeast genus Candida, preferably a strain of Candida boidinii, especially the Candida boidinii xylose isomerase disclosed by, e.g., Vongsuvanlert et al., 1988, Agric. Biol. Chem., 52(7): 1817-1824. The xylose isomerase may preferably be derived from a strain of Candida boidinii (Kloeckera 2201), deposited as DSM 70034 and ATCC 48180, disclosed in Ogata et al., Agric. Biol. Chem, 33: 1519-1520 or Vongsuvanlert et al., 1988, Agric. Biol. Chem, 52(2): 1519-1520.

In one embodiment the xylose isomerase is derived from a strain of Streptomyces, e.g., derived from a strain of Streptomyces murinus (U.S. Pat. No. 4,687,742); S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Pat. No. 4,351,903, U.S. Pat. No. 4,137,126, U.S. Pat. No. 3,625,828, HU patent no. 12,415, DE patent 2,417,642, JP patent no. 69,28,473, and WO 2004/044129 each incorporated by reference herein.

The xylose isomerase may be either in immobilized or liquid form. Liquid form is preferred.

Examples of commercially available xylose isomerases include SWEETZYME™ T from Novozymes NS, Denmark.

The xylose isomerase is added to provide an activity level in the range from 0.01-100 IGIU per gram total solids.

### Proteases

A protease may be added during hydrolysis in step ii), fermentation in step iii) or during simultaneous hydrolysis and fermentation. The protease may be any protease. In a preferred embodiment the protease is an acid protease of microbial origin, preferably of fungal or bacterial origin. An acid fungal protease is preferred, but also other proteases can be used.

Suitable proteases include microbial proteases, such as fungal and bacterial proteases. Preferred proteases are acidic proteases, i.e., proteases characterized by the ability to hydrolyze proteins under acidic conditions below pH 7.

Contemplated acid fungal proteases include fungal proteases derived from Aspergillus, Mucor, Rhizopus, Candida, Coriolus, Endothia, Enthomophtra, Irpex, Penicillium, Sclerotiumand Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae, such as the pepA protease; and acidic proteases from Mucor pusillus or Mucor miehei.

Contemplated are also neutral or alkaline proteases, such as a protease derived from a strain of Bacillus. A particular protease contemplated for the invention is derived from Bacillus amyloliquefaciens and has the sequence obtainable at Swissprot as Accession No. P06832. Also contemplated are the proteases having at least 90% identity to amino acid sequence obtainable at Swissprot as Accession No. P06832 such as at least 92%, at least 95%, at least 96%, at least 97%, at least 98%, or particularly at least 99% identity.

Further contemplated are the proteases having at least 90% identity to amino acid sequence disclosed as SEQ. ID. NO:1 in the WO 2003/048353 such as at 92%, at least 95%, at least 96%, at least 97%, at least 98%, or particularly at least 99% identity.

Also contemplated are papain-like proteases such as proteases within E.C. 3.4.22.* (cysteine protease), such as EC 3.4.22.2 (papain), EC 3.4.22.6 (chymopapain), EC 3.4.22.7 (asclepain), EC 3.4.22.14 (actinidain), EC 3.4.22.15 (cathepsin L), EC 3.4.22.25 (glycyl endopeptidase) and EC 3.4.22.30 (caricain).

In an embodiment the protease is a protease preparation derived from a strain of Aspergillus, such as Aspergillus oryzae. In another embodiment the protease is derived from a strain of Rhizomucor, preferably Rhizomucor mehei. In another contemplated embodiment the protease is a protease preparation, preferably a mixture of a proteolytic preparation derived from a strain of Aspergillus, such as Aspergillus oryzae, and a protease derived from a strain of Rhizomucor, preferably Rhizomucor mehei.

Aspartic acid proteases are described in, for example, Hand-book of Proteolytic Enzymes, Edited by A. J. Barrett, N. D. Rawlings and J. F. Woessner, Aca-demic Press, San Diego, 1998, Chapter 270). Suitable examples of aspartic acid protease include, e.g., those disclosed in R. M. Berka et al. Gene, 96, 313 (1990)); (R. M. Berka et al. Gene, 125, 195-198 (1993)); and Gomi et al. Biosci. Biotech. Biochem. 57, 1095-1100 (1993), which are hereby incorporated by reference.

Commercially available products include ALCALASE®, ESPERASE™ FLAVOURZYME™, PROMIX™, NEUTRASE®, RENNILASE®, NOVOZYM™ FM 2.0L, and NOVOZYM™ 50006 (available from Novozymes NS, Denmark) and GC106™ and SPEZYME™ FAN from Genencor Int., Inc., USA.

The protease may be present in an amount of 0.0001-1 mg enzyme protein per g DS, preferably 0.001 to 0.1 mg enzyme protein per g DS. Alternatively, the protease may be present in an amount of 0.0001 to 1 LAPU/g DS, preferably 0.001 to 0.1 LAPU/g DS and/or 0.0001 to 1 mAU-RH/g DS, preferably 0.001 to 0.1 mAU-RH/g DS.

### Pectinases

The pectinase may be any suitable pectinase such as pectate lyases.

Pectate lyases (also called polygalacturonate lyases): Examples of pectate lyases include pectate lyases that have been cloned from different bacterial genera such as Erwinia, Pseudomonas, Klebsiella and Xanthomonas, as well as from Bacillus subtilis (Nasser et al. (1993) FEBS Letts. 335:319-326) and Bacillus sp. YA-14 (Kim et al. (1994) Biosci. Biotech. Biochem. 58:947-949). Purification of pectate lyases with maximum activity in the pH range of 8-10 produced by Bacillus pumilus (Dave and Vaughn (1971) J. Bacteriol. 108:166-174), B. polymyxa (Nagel and Vaughn (1961) Arch. Biochem. Biophys. 93:344-352), B. stearothermophilus (Karbassi and Vaughn (1980) Can. J. Microbiol. 26:377-384), Bacillus sp. (Hasegawa and Nagel (1966) J. Food Sci. 31:838-845) and Bacillus sp. RK9 (Kelly and Fogarty (1978) Can. J. Microbiol. 24:1164-1172) have also been described. Any of the above, as well as divalent cation-independent and/or thermostable pectate lyases, may be used in practicing the invention. In preferred embodiments, the pectate lyase comprises the amino acid sequence of a pectate lyase disclosed in Heffron et al., (1995) Mol. Plant-Microbe Interact. 8: 331-334 and Henrissat et al., (1995) Plant Physiol. 107: 963-976. Specifically contemplated pectate lyases are disclosed in WO 99/27083 and WO 99/27084. Other specifically contemplated pectate lyases derived from Bacillus licheniformis is disclosed as SEQ ID NO: 2 in U.S. Pat. No. 6,284,524 (which document is hereby incorporated by reference). Specifically contemplated pectate lyase variants are disclosed in WO 02/006442, especially the variants disclosed in the Examples in WO 02/006442 (which document is hereby incorporated by reference).

Examples of commercially available alkaline pectate lyases include BIOPREP™ and SCOURZYME™ L from Novozymes NS, Denmark.

### Composition

In this aspect the invention relates to a composition comprising one or more trehalases. Examples of suitable trehalases can be found the “Trehalases” section above.

In an embodiment the composition further comprises one or more other carbohydrases, such as alpha-amylases. In a preferred embodiment the alpha-amylase is an acid alpha-amylase or a fungal alpha-amylase, preferably an acid fungal alpha-amylase.

The composition may comprise one or more carbohydrate-source generating enzymes, such as especially glucoamylases, beta-amylases, maltogenic amylases, pullulanases, alpha-glucosidases, or a mixture thereof.

In an embodiment the composition comprises enzymes selected from the group consisting of cellulolytic enzymes, such as cellulases, and/or hemicellulolytic enzymes, such as hemicellulases.

Examples of contemplated enzymes can be found in the “Enzymes” section above.

In another preferred embodiment the composition comprises one or more trehalases and further one or more fermenting organisms, such as yeast and/or bacteria. Examples of fermenting organisms can be found in the “Fermenting Organisms” section above.

### Use

In this aspect the invention relates to the use of trehalase in a fermentation process. In an embodiment one or more trehalases are used for increase the fermentation product yield.

### Transgenic Plant Material

In this aspect the invention relates to transgenic plant material transformed with one or more trehalase genes.

In one embodiment the invention relates to a transgenic plant, plant part, or plant cell which has been transformed with a polynucleotide sequence encoding a trehalase so as to express and produce the enzyme. The enzyme may be recovered from the plant or plant part, but in context of the present invention the plant or plant part containing the recombinant trehalase may be used in one or more of the methods or processes of the invention concerned and described above.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and corn.

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilisation of the invention are also considered plant parts, e.g., embryos, endosperms, aleurone and seeds coats.

Also included within the scope of the present invention are the progeny of such plants, plant parts, and plant cells.

The transgenic plant or plant cell expressing a trehalase may be constructed in accordance with methods well known in the art. In short, the plant or plant cell is constructed by incorporating one or more expression constructs encoding the trehalase into the plant host genome and propagating the resulting modified plant or plant cell into a transgenic plant or plant cell.

The expression construct is conveniently a nucleic acid construct which comprises a polynucleotide encoding trehalase operably linked with appropriate regulatory sequences required for expression of the polynucleotide sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the enzyme is desired to be expressed. For instance, the expression of the gene encoding trehalase may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, and the rice actin 1 promoter may be used (Franck et al., 1980, Cell 21: 285-294, Christensen et al., 1992, Plant Mo. Biol. 18: 675-689; Zhang et al., 1991, Plant Cell 3: 1155-1165). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588). Likewise, the promoter may inducible by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of an trehalase in the plant. For instance, the promoter enhancer element may be an intron which is placed between the promoter and the polynucleotide sequence encoding trehalase. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38) and can also be used for transforming monocots, although other transformation methods are often used for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated the expression construct are selected and regenerated into whole plants according to methods well-known in the art. Often the transformation procedure is designed for the selective elimination of selection genes either during regeneration or in the following generations by using, for example, co-transformation with two separate T-DNA constructs or site specific excision of the selection gene by a specific recombinase.

A method for producing trehalase in a plant would comprise: (a) cultivating a transgenic plant or a plant cell comprising a polynucleotide encoding trehalase under conditions conducive for production of the enzyme.

As mentioned above the transgenic plant material may be used in a method or process of the invention described above.

The transgenic plant is capable of expressing one or more trehalases in increased amounts compared to corresponding unmodified plant material.

### Modified Fermenting Organisms

In this aspect the invention relates to modified fermenting organisms transformed with a polynucleotide encoding trehalase, wherein the fermenting organism(s) is(are) capable of expressing trehalase at fermentation conditions.

In a preferred embodiment the trehalase is secreted into the fermenting medium. In a preferred embodiment the fermentation conditions are as defined according to the invention. In a preferred embodiment the fermenting organism is a microbial organism, such as yeast or filamentous fungus, or a bacterium. Examples of other fermenting organisms can be found the in “Fermenting Organisms” section.

A fermenting organism may be transformed with trehalase encoding genes using techniques well know in the art.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

### MATERIALS & METHODS

### Materials:

Trehalase: Porcine Trehalase purchased from Sigma (Cat # T8778).
Glucoamylase blend AG consisting of glucoamylase derived from Talaromyces emersonii disclosed as SEQ ID NO: 7 in WO 99/28448; glucoamylase derived from Trametes cingulata disclosed in SEQ ID NO: 2 in WO 2006/069289, and hybrid alpha-amylase consisting of Rhizomucor pusillus alpha-amylase with Aspergillus niger glucoamylase linker and SBD disclosed as V039 in Table 5 in WO 2006/069290 (all enzymes available from Novozymes NS, Denmark).
Yeast: RED STAR™ available from Red Star/Lesaffre, USA
In Example 1 corn mash from Adkins Energy, USA was used.
In Example 2 liquefied corm mash from Verasun Fort Dodge, Iowa, USA, was used.
In Example 3 backset from Hawkeye Fairbanks, Iowa, USA, and Verasun Energy was used.

### Equipment and Method Conditions Used in Example 2

Dionex BioLC Ion Chromatography System

CarboPac PA100 analytical column (4 mm inner diameter by 250 mm long) with CarboPac PA100 guard column

ED50 pulsed electrochemical detector with Au electrode

GP50 gradient pump

AS50 autosampler

Method Conditions: Flow rate was 0.80mL/min. Initial mobile phase composition was 10% deionized water, 90% 100 mM NaOH. 10 minutes after sample injection, the composition was linearly ramped from the initial composition to 100% of 100 mM NaOH+1M Na acetate over 25 minutes. The composition was held at 100% of 100 mM NaOH+1M Na acetate for 5 minutes, then changed to 60% deionized water, 40% 100 mM NaOH over 0.1 minutes. The composition was held there for the remaining 20 minutes of the sample run.

### Methods:

### Identity

The relatedness between two amino acid sequences or between two polynucleotide sequences is described by the parameter “identity”.

For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=1, gap penalty=3, windows=5, and diagonals=5.

For purposes of the present invention, the degree of identity between two polynucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

### SIGMA Enzymatic Assay for Trehalase

One SIGMA unit will convert 1.0 micro mol of trehalose to 2.0 micro mol of glucose per minutes at pH 5.7 at 37° C. (liberated glucose determined at pH 7.5).

### Glucoamylase Activity

Glucoamylase activity may be measured in Glucoamylase Units (AGU).

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

AMG incubation:

| | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

Color reaction:

| | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes NS, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes NS, Denmark, which folder is hereby included by reference.

### Acid Alpha-Amylase Activity

When used according to the present invention the activity of an acid alpha-amylase may be measured in AFAU (Acid Fungal Alpha-amylase Units) or FAU-F.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

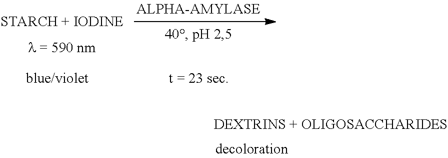

### Standard Conditions/Reaction Conditions:

Substrate: Soluble starch, approx. 0.17 g/L

Buffer: Citrate, approx. 0.03 M

Iodine (I2): 0.03 g/L

CaCl₂: 1.85 mM

pH: 2.50±0.05

Incubation temperature: 40° C.

Reaction time: 23 seconds

Wavelength: 590 nm

Enzyme concentration: 0.025 AFAU/mL

Enzyme working range: 0.01-0.04 AFAU/mL

A folder EB-SM-0259.02/01 describing this analytical method in more detail is available upon request to Novozymes NS, Denmark, which folder is hereby included by reference.

### Determination of FAU-F

FAU-F Fungal Alpha-Amylase Units (Fungamyl) is measured relative to an enzyme standard of a declared strength.

Reaction conditions

| | |
|---|---|
| Temperature | 37° C. |
| pH | 7.15 |
| Wavelength | 405 nm |
| Reaction time | 5 min |
| Measuring time | 2 min |

A folder (EB-SM-0216.02) describing this standard method in more detail is available on request from Novozymes NS, Denmark, which folder is hereby included by reference.

### Measurement of Cellulase Activity Using Filter Paper Assay (FPU Assay)

1. Source of Method
1.1 The method is disclosed in a document entitled “Measurement of Cellulase Activities” by Adney, B. and Baker, J. 1996. Laboratory Analytical Procedure, LAP-006, National Renewable Energy Laboratory (NREL). It is based on the IUPAC method for measuring cellulase activity (Ghose, T. K., Measurement of Cellulose Activities, Pure & Appl. Chem. 59, pp. 257-268, 1987.
2. Procedure
2.1 The method is carried out as described by Adney and Baker, 1996, supra, except for the use of a 96 well plates to read the absorbance values after color development, as described below.
2.2 Enzyme Assay Tubes:
2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.
2.3 Blank and Controls
2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.
2.4 Glucose Standards
2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:
   G1=1.0 mL stock+0.5 mL buffer=6.7 mg/mL=3.3 mg/0.5 mL
   G2=0.75 mL stock+0.75 mL buffer=5.0 mg/mL=2.5 mg/0.5 mL
   G3=0.5 mL stock+1.0 mL buffer=3.3 mg/mL=1.7 mg/0.5 mL
   G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.
2.5 Color Development
2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH2O in a 96-well plate. Each well is mixed, and the absorbance is read at 540 nm.
2.6 Calculations (Examples are Given in the NREL Document)
2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows: FPU/mL=0.37/enzyme dilution producing 2.0 mg glucose

### Protease Assay Method—AU(RH)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU-RH) is defined as the amount of enzyme which under standard conditions (i.e. 25° C., pH 5.5 and 10 min. reaction time) digests hemoglobin at an initial rate such that there is liberated per minute an amount of TCA soluble product which gives the same color with phenol reagent as one milliequivalent of tyrosine.

The AU(RH) method is described in EAL-SM-0350 and is available from Novozymes NS Denmark on request.

### Protease Assay Method (LAPU)

1 Leucine Amino Peptidase Unit (LAPU) is the amount of enzyme which decomposes 1 microM substrate per minute at the following conditions: 26 mM of L-leucine-p-nitroanilide as substrate, 0.1 M Tris buffer (pH 8.0), 37° C., 10 minutes reaction time.

LAPU is described in EB-SM-0298.02/01 available from Novozymes NS Denmark on request.

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

### EXAMPLES

### Example 1

### Fermentation of Liquefied Corn Mash With Trehalase

A fermentation study was conducted in which trehalase from Sigma Chemical Company (cat. #T8778) was added at 0.10 g protein/L (40 Sigma Units) to a conventional liquefied corn mash from Adkins Energy (30.2 wt % DS). Fermentation was carried out at 32° C. for 70 hours. 500 ppm of urea and 3 ppm of penicillin were added (final concentrations in 5.0 g mash samples, samples fermented in 15 mL conical plastic centrifuge tubes with small hole drilled at top for CO₂ release). Glucoamylase blend AG was added at time 0 at a dose of 0.37 AGU/g DS. RED STAR™ yeast was pitched into the fermentation at time 0 at an initial cell density of 30,000,000 cells/mL. The quantity of ethanol was measured by HPLC

The results of the study are displayed in FIG. 1 showing that addition of trehalase after 0, 24 and 48 hours, respectively, in a 70 hour fermentation leads to increased ethanol yield.

### Example 2

### Trehalase Treatment

A fermentation study was carried out as described in Examples 1, except that conventional liquefied corn mash was from Verasun Fort Dodge with 32 wt % DS and trehalase was added at time 0 and after 63 hours.

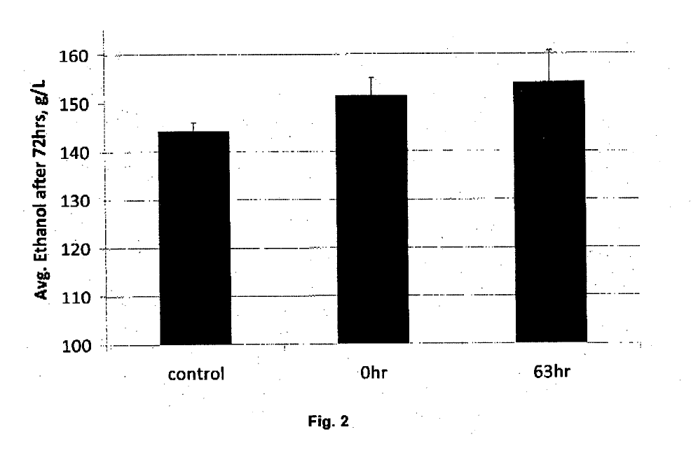

The results of the study are displayed in FIG. 2 showing that addition of trehalase after 0 and 63 hours, respectively, in a 70 hour fermentation leads to increased ethanol yield.

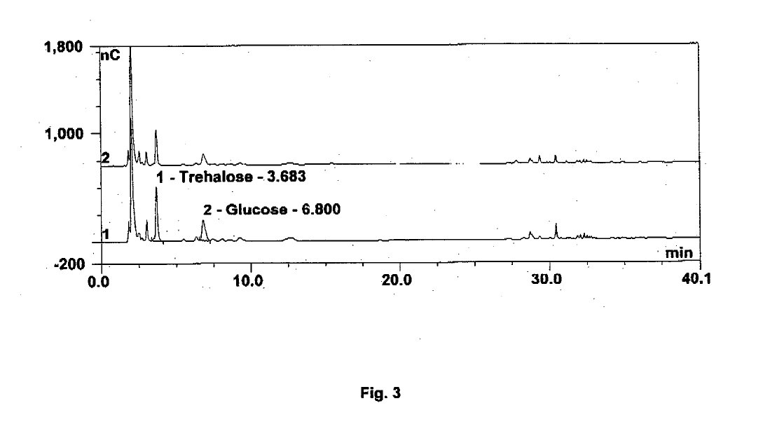

FIG. 3 shows a chromatogram from an ion chromatographic system which illustrates the reduction in the peak for trehalose observed upon addition of trehalase to the fermentation of the corn mash. The top curve is for the mash treated with trehalase, and the bottom curve is for the untreated mash.

### Example 3

### Fermentation of Backset With Trehalase

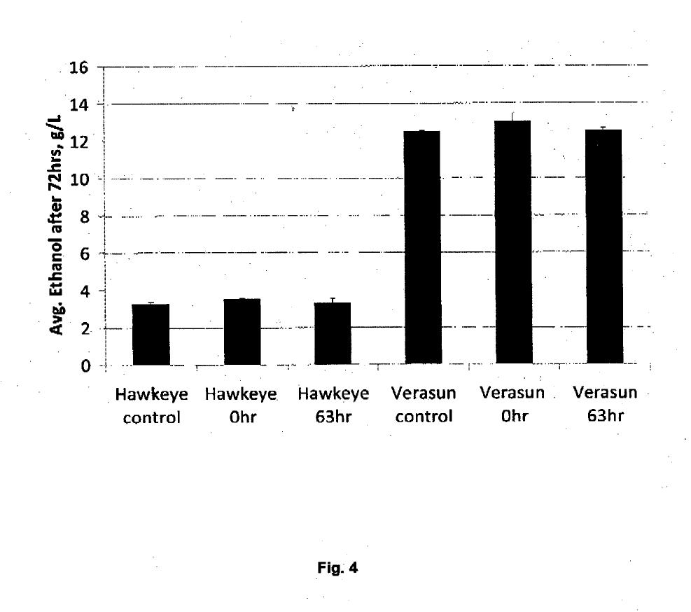

A fermentation study was carried out as described in Examples 1, except that trehalase was added at 0.20 g protein/L (80 Sigma Units) to samples of backset from Verasun Energy (6.7 wt. % DS) and Hawkeye Fairbanks (5.8 wt. % DS), respectively. Trehalase was added at either the start of the fermentation or after 63 hours. The quantity of ethanol was measured by HPLC. The results of the study are displayed in FIG. 4 showing that addition of trehalase is more beneficial to the ethanol yield if added at the start of the fermentation compared to after 63 hours.

1-20. (canceled)

21. A method of producing a fermentation product, comprising fermenting a sugar in a fermentation medium using a fermenting organism, wherein one or more trehalases are present in the fermentation medium.

22. The method of claim 21, wherein the one or more trehalases are added before and/or during fermentation.

23. The method of claim 21, wherein the fermentation product is ethanol.

24. The process of claim 21, further comprising recovering the fermentation product by distillation after fermentation.

25. A process of producing a fermentation product, comprising:
(a) liquefying a starch-containing material;
(b) saccharifying the liquefied material; and
(c) fermenting according to the method of claim 21.

26. A process of producing a fermentation product, comprising:
(a) liquefying a starch-containing material;
(b) saccharifying the liquefied material; and
(c) fermenting with one or more fermenting organisms;
wherein backset subjected to trehalase treatment is adding during or before step (a).

27. The process of claim 26, further comprising, prior to step (a), the steps of:
x) reducing the particle size of starch-containing material; preferably by milling
y) forming a slurry comprising the starch-containing material and water.

28. The process of claim 26, wherein steps (b) and (c) are carried out simultaneously.

29. A process of producing a fermentation product from starch-containing material, comprising the steps of:
(a) saccharifying starch-containing material at a temperature below the initial gelatinization temperature of said starch-containing material; and
(b) fermenting according to the method of claim 21.

30. The process of claim 29, wherein steps (a) and (b) are carried out simultaneously.

31. A process of producing a fermentation product, comprising:
(a) pre-treating a lignocellulose-containing material;
(b) hydrolyzing the lignocellulose-containing material; and
(c) fermenting according to the method of claim 21.

32. The process of claim 31, wherein steps (b) and (c) are carried out as HHF, SSF, or SHF.

33. The process of claim 31, wherein fermentation is initiated and carried out at:
a) a fermentation organism cell count in the range of 10-250×10¹⁰ cells per L fermentation medium; or
b) a fermentation organism concentration in the range of 2-50 g TS fermenting organism per L fermentation medium.

34. The process of claim 31, wherein the fermenting organisms are recovered from the fermentation product by filtration using a filter press or by centrifugation.
